(19) United States
(12) Patent Application Publication
(10) Pub. No.: US 2022/0273528 A1
(43) Pub. Date: Sep. 1, 2022

(54) SOLID HAIR CONDITIONING COMPOSITIONS

(71) Applicant: Henkel AG & Co. KGaA, Duesseldorf (DE)

(72) Inventors: Soeren SCHEELE, Pinneberg (DE); Petra WESTPHAL, Neu Wulmstorf (DE); Manuela METTE, Kleinfeld (DE); Thomas SCHROEDER, Hamburg (DE)

(73) Assignee: Henkel AG & Co. KGaA, Duesseldorf (DE)

(21) Appl. No.: 17/626,116

(22) PCT Filed: Apr. 29, 2020
(86) PCT No.: PCT/EP2020/061823

§ 371 Date: Jan. 10, 2022

(30) Foreign Application Priority Data
Jul. 10, 2019 (DE) 10 2019 210 152.6

(51) Int. Cl. A61K 8/02; A61K 8/92; A61K 8/34; A61K 8/86; A61K 8/49; A61K 8/41; A61K 8/73; A61Q 5/12

(52) U.S. Cl. CPC A61K 8/0229; A61K 8/922; A61K 8/345; A61K 8/86; A61K 8/4946; A61K 8/416; A61K 8/732; A61Q 5/12

(57) ABSTRACT

The present disclosure provides a solid hair conditioning composition with a low water content. The solid hair conditioning composition of the present disclosure comprises: (a) from about 10 wt % to about 70 wt % of at least one fat or oil having a melting point above about 20° C., (b) from about 3 wt % to about 50 wt % of at least one cationic surfactant, and (c) from about 0.5 wt % to about 20 wt % of a polyhydric alcohol and/or polyethylene glycol, wherein the water content is less than about 20 wt %.

### CROSS-REFERENCE TO RELATED APPLICATION

This application is a U.S. National-Stage entry under 35 U.S.C. § 371 based on International Application No. PCT/EP2020/061823, filed Apr. 29, 2020, which was published under PCT Article 21(2) and which claims priority to German Application No. 102019210152.6, filed Jul. 10, 2019, which are all hereby incorporated in their entirety by reference.

### TECHNICAL FIELD

The present disclosure relates to a solid hair conditioning composition with a low water content. In particular, the present disclosure relates to a solid hair conditioning composition that is mixed with water when used. The present disclosure also relates to processes for the preparation of the solid cosmetic compositions and their use for conditioning keratin fibres.

### BACKGROUND

Products comprising surfactants for conditioning mainly human hair, but also the skin of the human body, have been known for a long time and are offered on the market mainly in liquid or pasty form in suitable packaging. End users remove the required quantity of product from the packaging and dispose of it after emptying. Compared to products that are sold in solid form, such products offer the user the advantage of simple and fast handling, which is why they dominate the market today. However, this advantage is achieved by accepting certain disadvantages, which are discussed below. In most cases, the packaging of the described products in liquid or pasty form is made of non-recyclable plastic, which is a serious environmental problem in view of the constantly growing amount of plastic waste.

Another problem is that existing products typically contain higher amounts of water or water/solvent blends, resulting in higher volume products and, from a transport point of view, possibly higher importance, a relatively high weight. This is disadvantageous for several reasons. In times of increasing water scarcity, resources should be saved. Also of importance from an environmental and cost point of view is an undesirable, increased volume of transport that goes hand-in-hand with large-volume heavy products. Another interesting point is that worldwide travel activity is steadily increasing. Consumers are therefore increasingly interested in cosmetic products that are easy to transport due to their low weight and volume. This is particularly relevant with regard to air travel, since larger containers comprising liquids are generally excluded from being carried in the aircraft cabin, so that a person travelling only with hand luggage often finds themselves in the situation of not being able to take their preferred product selection with them or having to transfer it into smaller containers first, due to the cosmetic products dominating the market today, which can again be accompanied by an increasing volume of packaging material.

The provision of alternative product forms with lower water content, included in space-saving, more environmentally friendly packaging such as recyclable packaging, is therefore an important objective in the formulation of improved, contemporary and sustainable cosmetic products.

Solid conditioner compositions have been known for some time and occupy a market niche. Although they have a very low water or solvent content in general and are often packaged with little material input, many people find them uncomfortable to handle because a piece of conditioner that has already been started is difficult to transport, dissolves when it is near a shower or bathtub or next to the washbasin, which is also inefficient, and makes the washbasin or other storage location look unattractive due to conditioner residues, and because conditioner pieces have a tendency to slip out of the user's hand.

Another drawback with known solid conditioner formulations, especially with rather small conditioner pieces, is that it takes some time to separate enough from the conditioner piece to achieve the de-sired amount of conditioning effect. This is usually undesirable for one of the users because of the additional time required, on the other hand it can be accompanied by a higher water consumption in body care, since many users do not switch off the water flow of the shower or tap during conditioning. From this point of view, it does not make sense to market conditioner formulations known from individual application portions, in particular in miniaturized form of a known conditioner piece, since their dissolution is too slow since the formulations of these conditioners are not optimized for marketing in individual application portions.

There is a need to develop a solid hair conditioning composition with a low water content, which has excellent care properties, and which can be formulated in single-use doses, or as multi-doses, for example as a stick or a bar. Furthermore, there is a need to develop products and associated processes that avoid or reduce the need for single-use plastic packaging. In their intensive research efforts, the inventors have established several measures that can contribute to this suitability.

### BRIEF SUMMARY

Solid hair conditioning compositions, methods of preparing the same, and methods for treating hair with the same are provided herein. In an embodiment, a solid hair conditioning composition includes

(a) from about 10 wt % to about 70 wt % of at least one fat or oil having a melting point above about 20° C.,
(b) from about 3 wt % to about 50 wt % of at least one cationic surfactant, and
(c) from about 0.5 wt % to about 20 wt % of at least one polyhydric alcohol and/or polyethylene glycol,
wherein the water content is less than about 20 wt %, and wherein all amounts are based on the total weight of the solid hair conditioning composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description. Unless otherwise indicated, all amounts are based on total weight of the solid hair conditioning composition.

The present disclosure provides:

1. A solid hair conditioning composition, comprising
(a) from about 10 wt % to about 70 wt % of at least one fat or oil having a melting point of 20° C. or higher,
(b) from about 3 wt % to about 50 wt % of a cationic surfactant, and
(c) from about 0.5 wt % to about 20 wt % of a polyhydric alcohol and/or polyethylene glycol,
wherein the water content is less than about 20 wt %.
2. The solid hair conditioning composition according to point 1, wherein component (a) comprises a vegetable oil.
3. The solid hair conditioning composition according to point 1, wherein component (a) comprises a vegetable butter.
4. The solid hair conditioning composition according to point 1, wherein component (a) is selected from Butyrospermum parkii (Shea) Butter, Theobroma cacao (Cocoa) Seed Butter, Cocos nucifera (Coconut) Oil, Elaeis guineensis (Palm) Kernel Oil, Japan Wax, Synthetic Japan Wax, Ricinus communis (Castor) Seed Oil, Simmondsia chinensis (Jojoba) Butter and mixtures thereof.
5. A solid hair conditioning composition according to point 1, 3 or 4, wherein component (a) comprises Shea Butter (INCI designation: Butyrospermum parkii (Shea) Butter).
6. A solid hair conditioning composition according to point 1, wherein component (a) consists of Shea Butter (INCI designation: Butyrospermum parkii (Shea) Butter).
7. A solid hair conditioning composition according to any one of the preceding points, wherein the content of component (a) is from about 10 wt % to about 50 wt %, based on the total weight of the solid hair conditioning composition, preferably from about 10 wt % to about 40 wt %.
8. A solid hair conditioning composition according to any one of the preceding points, wherein the content of component (a) is from about 10 wt % to about 30 wt %.
9. The solid hair conditioning composition according to any one of the preceding points, comprising as cationic surfactant (b) at least one compound selected from the group of:
i. alkylquats,
ii. esterquats,
ii. quaternary imidazolines,
iv. amidoamines and/or cationized amidoamines and
v. mixtures thereof.
10. A solid hair conditioning composition according to point 9, comprising at least one cationic surfactant b) from group i.
11. A solid hair conditioning composition according to point 10, comprising C8-C30-alkyl-tri-C1-C4-alkylammonium salts, preferably C8-C24-alkyltrimethylammonium salts, particularly preferably lauryltrimethylammonium salts, cetyltrimethylammonium salts, stearyltrimethylammonium salts, behentrimethylammonium salts and/or mixtures thereof, in particular the chloride, methosulfate and/or ethosulfates of these cationic surfactants.
12. A solid hair conditioning composition according to any one of the preceding points, comprising cationic surfactant salts known under the INCI name “Cetrimonium” and/or “Behentrimonium”, preferably cetrimonium chloride and/or behentrimonium chloride.
13. A solid hair conditioning composition according to any one of the preceding points, comprising at least one cationic surfactant b) from group iii, in particular Quaternium-87.
14. A solid hair conditioning composition according to point 9, comprising as cationic surfactant (b): i) cetrimonium chloride and/or behentrimonium chloride; and iii) Quaternium-87.
15. The solid hair conditioning composition according to any one of the preceding points, wherein the content of the component (b) is from about 3 wt % to about 20 wt %, based on the total weight of the solid hair conditioning composition, preferably from about 3 wt % to about 10 wt %, more preferably from about 3 wt % to about 8 wt %, yet more preferably from about 3 wt % to about 5 wt %.
16. The solid hair conditioning composition according to any one of the preceding points, wherein the polyhydric alcohol is selected from glycerin, mannitol, isomalt, lactitol, sorbitol, xylitol, threitol, erythritol, arabitol, 1,2-propylene glycol, 1,3-butylene glycol, 1,6-hexanediol, dipropylene glycol, diglycerol, and mixtures thereof.
17. The solid hair conditioning composition according to any one of the preceding points, wherein the polyhydric alcohol is a mixture of glycerin and a glycol.
18. The solid hair conditioning composition according to any one of the preceding points, wherein the polyhydric alcohol is a mixture of glycerin and one or more of 1,2-propylene glycol, 1,3-butylene glycol and dipropylene glycol.
19. The solid hair conditioning composition according to any one of the preceding points, wherein the polyhydric alcohol is a mixture of glycerin and 1,2-propylene glycol.
20. The solid hair conditioning composition according to any one of points 1 to 16, wherein component c) consists of glycerin.
21. The solid hair conditioning composition according to any one of points 1 to 19, wherein component c) includes a polyethylene glycol; suitably a polyethylene glycol having a molecular weight of from about 300 to about 5,000,000.
22. The solid hair conditioning composition according to any one of points 1 to 20, which excludes a polyethylene glycol.
23. The solid hair conditioning composition according to any one of the preceding points, wherein the content of component (c) is from about 1% to about 15% by weight, based on the total weight of the solid hair conditioning composition, preferably from about 3 wt % to about 15 wt %, more preferably from about 5 wt % to about 15 wt %.
24. The solid hair conditioning composition according to any one of the preceding points, further comprising from about 10 to about 60% by weight of at least one polysaccharide, preferably from about 20 to about 60% by weight of at least one polysaccharide d).
25. The solid hair conditioning composition according to point 24, comprising as polysaccharide d) di) at least one starch and/or dii) at least one modified starch and/or diii) at least one dextrin.
26. The solid hair conditioning composition according to point 24, comprising as polysaccharide d) di) at least one starch and diii) at least one dextrin.
27. The solid hair conditioning composition according to point 26, wherein the weight ratio of di) the or each starch to diii) the or each dextrin ranges from about 10:1 to about 60:1, more preferably from about 20:1 to about 60:1, yet more preferably from about 30:1 to about 50:1.
28. The solid hair conditioning composition according to any of points 24 to 27, comprising at least one starch derived from natural sources.
29. The solid hair conditioning composition according to any of points 24 to 28, comprising at least one of corn, rice, potato or tapioca starch.
30. The solid hair conditioning composition according to any of points 24 to 29, wherein the polysaccharide d) comprises a corn starch.
31. The solid hair conditioning composition according to any of points 24 to 30, wherein the polysaccharide d) comprises a dextrin.
32. The solid hair conditioning composition according to any of points 24 to 31, wherein the dextrin is maltodextrin.
33. The solid hair conditioning composition according to any of points 24 to 25 or 28 to 33, wherein the polysaccharide comprises d) a modified starch.
34. The solid hair conditioning composition according to point 33, wherein the modified starch is hydroxypropyl starch phosphate.
35. The solid hair conditioning composition according to point 24, wherein the polysaccharide d) comprises or consists of a corn starch; maltodextrin; and hydroxypropyl starch phosphate.
36. The solid hair conditioning composition according to any of points 24 to 35, wherein the corn starch is Zea mays (Corn) Starch.
37. The solid hair conditioning composition according to any of points 24 to 36, comprising from about 10 to about 60%, preferably from about 20 to about 60%, more preferably from about 30 to about 55% and particularly preferably, from about 35 to about 55% by weight of component d) based on the total weight of the cosmetic composition.
38. The solid hair conditioning composition according to any one of the preceding points, further comprising potassium carbonate, ammonium carbonate or sodium bicarbonate, preferably sodium bicarbonate.
39. The solid hair conditioning composition according to any one of the preceding points, further comprising at least one fatty alcohol, at least one fatty acid or salt thereof, and/or at least one fatty alcohol alkoxylate.
40. The solid hair conditioning composition according to any one of the preceding points, further comprising a cationic polymer.
41. The solid hair conditioning composition according to point 40, wherein the cationic polymer is a polysaccharide polymer obtainable from guar, cassia and/or inulin.
42. The solid hair conditioning composition according to any one of the preceding points, comprising water in an amount up to about 15% by weight (based on the weight of the composition).
43. The solid hair conditioning composition according to any one of the preceding points, comprising water in an amount up to about 12% by weight (based on the weight of the composition).
44. The solid hair conditioning composition according to any one of the preceding points, comprising water in an amount up to about 10% by weight (based on the weight of the composition).
45. The solid hair conditioning composition according to any one of the preceding points, comprising water in an amount up to about 8% by weight (based on the weight of the composition).
46. The solid hair conditioning composition after one of the preceding points in the form of a bar, a stick or a monoportion.
47. The solid hair conditioning composition according to point 46 in the form of a bar or a stick.
48. The solid hair conditioning composition according to point 47, having a Shore A hardness value of from about 10 to about 30.
49. The solid hair conditioning composition according to point 48, having a Shore A hardness value of from about 15 to about 25.
50. The solid hair conditioning composition according to point 46 in the form of a monoportion.
51. The solid hair conditioning composition according to point 50, having a Shore AO hardness value of from about 15 to about 35.
52. The solid hair conditioning composition according to point 51, having a Shore AO hardness value of from about 20 to about 30.
53. The solid hair conditioning composition according to any one of the preceding points for the cleansing and/or care of human skin and/or hair.
54. The solid hair conditioning composition according to one of the preceding points in the form of a hair conditioner.
55. The solid hair conditioning composition according to any one of the preceding points in the form of a leave-on or rinse-off hair conditioner.
56. A method for preparing the solid hair conditioning composition of the present disclosure, comprising:
a) mixing all the components with sufficient heating and agitation to ensure all the components are melted and/or uniformly dispersed,
b) pouring the resultant liquid into a mold or directly into packaging, and
c) allowing the liquid to cool into a solid.
57. The method according to point 56, wherein the mixing step (i) comprises using a batch mixer to mix the components in one or more batches with sufficient heating and agitation to ensure all the components are melted and/or uniformly dispersed.
58. A method for preparing the solid hair conditioning composition of the present disclosure, comprising:
a) mixing all the components with sufficient heating and agitation to ensure all the components are melted and/or uniformly dispersed,
b) injecting the resultant liquid into a mold or directly into packaging, and
c) allowing the liquid to cool into a solid.
59. A method for preparing the solid hair conditioning composition of the present disclosure, comprising:
a) using a batch mixer to mix all the components in one or more batches with sufficient heating and agitation to ensure all the components are melted and/or uniformly dispersed to form a liquid,
b) optionally extruding the liquid,
c) transferring the liquid to a piston injection unit,
d) injecting the liquid into a mold or directly into packaging, and
e) allowing the liquid to cool into a solid.
60. The method according to any one of points 56 to 59, comprising:
(i) adding two or more ingredients to a drum;
(ii) melting the ingredients at a suitable temperature until the mixture is homogeneous;
(iii) maintaining the drum at a temperature ranging from about 70° C. to about 95° C.;
(iv) adding further ingredients in one or more further batches to the drum; and
(v) repeating steps (2) and (3) after each batch addition.
61. A solid hair conditioner obtained by a method as defined in any one of point 56 to 60.
62. A method for using the solid hair conditioning composition according to any of point 1 to 55 comprising: mixing the solid hair conditioning composition with water and applying to the hair, or applying the solid hair conditioning composition directly to wet hair.

The solid hair conditioning composition according to the present disclosure has an excellent appearance, a high hardness and good care properties.

The compositions as contemplated herein are solid at 25° C. Solid compositions within the meaning of this application are three-dimensional, dimensionally stable structures which are not liquid or gaseous, i.e. which maintain their external shape even without a receptacle surrounding them. However, the term “solid” says nothing about density or elasticity or other physical properties, so that jelly, aspic, butter etc. can also be solid as contemplated herein, as long as they are dimensionally stable at 25° C.

Such a formulation offers the right properties for individual application portions, in particular as regards their dissolution behaviour during use. The high concentrations of the active substances in such a composition are associated with the advantages that few resources are consumed during production and transport and the products can be transported without great effort or restrictions even after they have reached the consumer's hands via the trade, whether to the gym or during a flight.

The ingredients for inclusion in the compositions of the present disclosure, as defined above and in the claims, will now be described in more detail.

### (A) Solid Fat or Oil

The solid hair conditioning composition of the present disclosure comprise as component (a) at least one fat or oil having a melting point of 20° C. or higher. In the context of a solid fat or oil, “solid” means the compound has a melting point of 20° C. or higher. Suitably, the or each fat or oil has a melting point of 25° C. or higher, or 30° C. or higher. Preferably, component (a) is a naturally occurring solid fat or oil, or mixture thereof.

These are caring substances that help to keep both the skin and hair structure healthy. The defined concentration range makes it possible to use this care effect while at the same time excluding a noticeable greasiness after application of an appropriate composition. Naturally occurring raw materials have the advantage that they grow back and can therefore be used sustainably. This aspect is also becoming increasingly important for many users.

Solid fats and oils include fatty acids (triglycerides); both naturally-derived products and hydrogenated derivatives thereof. Also included are fatty acids esterified with glycerin to yield mono-, di-, and triglycerides.

Preferably, component (a) is a solid vegetable oil and/or solid vegetable butter.

As already mentioned, naturally occurring raw materials have the advantage that they grow again and can therefore be used sustainably. This aspect is also becoming increasingly important for many users. In addition, some vegetable oils or butters, especially when obtained gently at low temperatures, are extremely potent skin and hair care products, as they also contain a variety of certain secondary ingredients, such as vitamins.

It has been found that plant butter with a melting range of from about 20° C. to about 35° C. is particularly suitable for incorporation into the inventive solid hair conditioning compositions.

Particularly preferred are plant butters with a melting point in the range from about 20° C. to about 35° C., such as Shea Butter (INCI designation: Butyrospermum parkii (Shea) Butter), Mango Butter (INCI designation: Butyrospermum parkii (Shea) Butter), Mangifera indica (Mango) Seed Butter), Murumuru Butter (INCI designation: Astrocaryum murumuru Seed Butter), Cocoa Butter (INCI designation: Theobroma cacao (Cocoa) Seed Butter) and/or Cupuacu Butter (INCI designation: Theobroma grandiflorum Seed Butter), Cocos nucifera (Coconut) Oil, Elaeis guineensis (Palm) Kernel Oil, Japan Wax, Synthetic Japan Wax, Ricinus communis (Castor) Seed Oil, Simmondsia chinensis (Jojoba) Butter and mixtures thereof.

Preferred solid fats are Cupuacu butter (INCI designation: Theobroma grandiflorum Seed Butter) and/or Shea butter (INCI designation: Butyrospermum parkii (Shea) Butter), and Shea butter (INCI designation: Butyrospermum parkii (Shea) Butter) is particularly preferred.

The fats and oils used as component (a) are not fatty alcohols. Suitably, the composition of the present disclosure does not comprise any fatty alcohol. Alternatively, the composition of the present disclosure comprises component (a) and at least one fatty alcohol.

Component (a) is present in an amount of from about 10 wt % to about 70 wt %, preferably from about 10 wt % to about 50 wt %. Suitably, component (a) is present in an amount ranging from about 40 wt % to about 50 wt %, based on the total weight of the solid hair conditioning composition. Alternatively and preferably, the component (a) is present in an amount ranging from about 10 wt % to about 30 wt %, based on the total weight of the solid hair conditioning composition.

### (b) Cationic Surfactant

The solid hair conditioning composition disclosed herein comprises b) at least one cationic surfactant.

Cationic surfactants carry a positive charge in their hydrophilic part. This positive charge causes the surfactant molecules to attach themselves to the negatively charged skin and hair surface. In this way they neutralize the charge, prevent hair from flying, have a smoothing effect, increase hair shine and improve wet combability. Primarily they are used in conditioners, hair conditioners and cures, less often in shampoos. In addition, cationic surfactants have a co-conservative effect in cosmetic products due to their bactericidal effect, i.e. their bacterial inhibitory effect.

In principle, all cationic surfactants suitable for use on the human body are suitable as cationic surfactants in compositions as contemplated herein. These are exemplified by at least one water-solubilizing cationic group, such as, for example, a quaternary ammonium group, or by at least one water-solubilizing cationizable group, such as, for example, an amine group, and furthermore at least one lipophilic alkyl group having from about 6 to about 30 carbon atoms, or also by at least one imidazole group or at least one imidazylalkyl group.

In general, cationic surfactants are divided into groups according to their structural characteristics. Cationic surfactants for use in the present disclosure may be chosen from at least one of the groups alkylquats, esterquats, quaternary imidazolines, amidoamines and/or cationized amidoamines.

Thus, the present disclosure therefore also concerns a solid cosmetic composition as described above, comprising as cationic surfactant (b) at least one compound selected from one of the following groups:

i. Alkylquats,
ii. Esterquats,
iii. quaternary imidazoline,
iv. Amidoamines and/or cationized amidoamines and
v. Mixtures of them.

These specifically named cationic surfactants have shown a particularly pleasant conditioning effect in the inventive compositions.

Particularly preferred inventive compositions comprising as cationic surfactants a)
Quaternary ammonium compounds (alkylquats) having at least one C8-C24-alkyl radical,
Esterquats and
Amidoamines each having at least one C8-C24-acyl radical
and mixtures thereof.

Particularly preferred quaternary ammonium compounds with at least one C8-C24 alkyl radical are ammonium halides, especially chlorides, and ammonium alkyl sulphates, such as methosulphates or ethosulphates, such as C8-C24 alkyltrimethylammonium chlorides, C8-C24 dialkyldimethylammonium chlorides and C8-C24 trialkylmethylammonium chlorides, e.g. cetyltrimethylammonium chloride, stearyltrimethylammonium chloride, distearyl dimethylammonium chloride, lauryldimethylammonium chloride, lauryldimethylbenzylammonium chloride and tricetylmethylammonium chloride, and the imidazolium compounds known under the INCI names Quaternium-27, Quaternium-83, Quaternium-87 and Quaternium-91. The alkyl chains of the surfactants mentioned above preferably have from about 8 to about 24 carbon atoms. Quaternium-87 is particularly preferred.

Esterquats are cationic surfactants which contain both at least one ester function and at least one quaternary ammonium group as structural element and furthermore at least one C8-C24 alkyl radical or C8-C24 acyl radical. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed under the trademarks Stepantex®, Dehyquart® and Armocare®. N,N-bis(2-palmitoyloxyethyl)dimethylammonium chloride, distearoylethyl dimonium methosulphates and distearoylethyl hydroxyethylmonium methosulphates are preferred examples of such esterquats.

Alkylamidoamines are usually prepared by amidation of natural or synthetic C8-C24 fatty acids and fatty acids with di-(C1-C3)alkylaminoamines. Compounds from this substance group which are particularly suitable as contemplated herein are, for example, those known under the INCI names stearamidopropyl dimethylamine, Bis-(Isostearoyl/Oleoyl Isopropyl) Dimonium Methosulfate, behenamidopropyl dimethylamine and/or brassicamidopropyl dimethylamine. Stearamidopropyl dimethylamine and Bis-(Isostearoyl/Oleoyl Isopropyl) Dimonium Methosulfate are particularly preferred.

The present disclosure also concerns, in a preferred form, a solid cosmetic composition as described above, comprising at least one cationic surfactant b) from group i, preferably C8-C30-alkyl-Tri-C1-C4-alkylammonium salts and cationic surfactant salts known in particular under the INCI designation “Cetrimonium” and/or “Behentrimonium”, for example cetrimonium chloride and/or behentrimonium chloride.

These specifically named cationic surfactant salts have shown a conditioning effect in the compositions as contemplated herein which is particularly perceived as pleasant.

Compositions preferred as contemplated herein contain at least one cationic surfactant b) in a total amount of from about 0.1 to about 15% by weight, preferably from about 2 to about 10% by weight, more preferably from about 2 to about 8% by weight, particularly preferably from about 2 to about 6% by weight, based in each case on the weight of the composition.

The compositions as contemplated herein can be cleaning agents, particularly shampoos, as well as conditioners. When formulated as shampoos, the composition disclosed herein:
comprises at least one cationic surfactant, preferably in a total amount of from about 0.1 to about 2 wt. %, more preferably from about 0.2 to about 1 wt. % and more preferably from about 0.3 to about 0.5 wt. %, in each case based on the weight of the composition, and
at least one further surfactant selected from anionic, amphoteric, zwitterionic and/or nonionic surfactants, preferably in a total amount of from about 1 to about 40% by weight, more preferably from about 2 to about 35% by weight and more preferably from about 3 to about 30% by weight, each based on the weight of the composition.
   (c) Polyhydric Alcohol and/or Polyethylene Glycol

According to the present disclosure, the solid hair conditioning composition comprises as component c) a polyhydric alcohol and/or polyethylene glycol.

Polyhydric alcohols are predominantly well tolerated by the skin and as solvents ensure that the solid cosmetic compositions available with them do not become too solid or too difficult or slowly soluble. In the defined concentration range, these advantageous properties are particularly noticeable.

The polyhydric alcohol may be selected from glycerin, alditols such as mannitol, isomalt, lactitol, sorbitol and xylitol, threit, erythritol and arabitol, glycols such as 1,2-propylene glycol, 1,3-butylene glycol, 1,6-hexanediol, dipropylene glycol, diglycerol, and mixtures thereof. Suitably, the polyhydric alcohol may be a mixture of glycerin and a glycol, for example a mixture of glycerin and one or more of 1,2-propylene glycol, 1,3-butylene glycol and dipropylene glycol. Preferably, the polyhydric alcohol is a mixture of glycerin and 1,2-propylene glycol. Alternatively, the polyhydric alcohol may consist of glycerin.

When a mixture of glycerin and glycols selected from 1,2-propylene glycol, 1,3-butylene glycol, 1,6-hexanediol are used, the glycerin is present in excess (by weight) relative to the glycol component. Preferably, the weight ratio of glycerin to glycol ranges from about 5:1 to about 30:1, more preferably from about 5:1 to about 20:1, yet more preferably from about 10:1 to about 20:1.

In addition to, or instead of, the polyhydric alcohol, component c) may include a polyethylene glycol; suitably a polyethylene glycol having a molecular weight of from about 300 to about 5,000,000.

The content of the component (c) in the solid hair conditioning composition disclosed herein is preferably from about 10% to about 15% by weight, based on the total weight of the solid hair conditioning composition, preferably from about 3 wt % to about 15 wt %, more preferably from about 5 wt % to about 15 wt %.

In the defined concentration range, the advantageous properties described above are even more pronounced.

### Water Content

The solid hair conditioning composition of the present disclosure comprises less than about 20 wt % of water, preferably less than about 15 wt % of water, more preferably less than about 10 wt % of water, yet more preferably less than about 8 wt % water.

### Fatty Compounds

The solid hair conditioning compositions disclosed herein may further comprise as an optional component at least one saturated or unsaturated, branched or unbranched C8-C30 alcohol.

Examples of suitable fatty alcohols include C12-13 alcohols, C12-15 alcohols, C12-16 alcohols, C14-22 alcohols, C16-17 isoalcohols, C20-22 alcohols, arachidyl alcohol, cetyl alcohol, cetearyl alcohol, myristyl alcohol, behenyl alcohol, hexyldecycloctadecanol, tetradecyleicosanol, coconut alcohol, isocetyl alcohol, isostearyl alcohol, stearyl alcohol and lauryl alcohol. Preferably, the fatty alcohol may be selected from the group of arachidyl alcohol, cetyl alcohol, cetearyl alcohol, myristyl alcohol, behenyl alcohol, hexyldecycloctadecanol, tetradecyleicosanol, coconut alcohol, isocetyl alcohol, isostearyl alcohol, stearyl alcohol and lauryl alcohol. More preferably, the fatty alcohol may be cetyl alcohol, stearyl alcohol, or mixtures thereof.

The solid hair conditioning compositions disclosed herein may further comprise as an optional component saturated or unsaturated, branched or unbranched C8-C30 carboxylic acids and/or their salts, preferably C10-C22 carboxylic acids and/or their salts and in particular coconut acids, lauric acid, myristic acid, palmitic acid, stearic acid, behenic acid, oleic acid and mixtures thereof and/or the salts of these acids. Stearic acid and palmitic acid and/or the salts of these acids, in particular stearic acid and palmitic acid, are particularly suitable.

Further fatty compounds that may be incorporated as optional components are those having a melting point of about 25° C. or higher and that are selected from alkoxylated (e.g. ethoxylated or propoxylated) fatty alcohols having from about 12 to about 22 carbon atoms in the alkyl chain. Suitable examples include ethylene glycol cetyl ether, polyoxyethylene (2) stearyl ether, polyoxyethylene (4) cetyl ether, and mixtures thereof.

The total amount of fatty compounds included in the compositions disclosed herein (other than the fatty fat or oil component a) ranges from about 0.1 to about 20% by weight, preferably from about 1 to about 15% by weight, particularly preferably from about 5 to about 15% by weight; in each case based on the weight of the composition.

Preferably, the total amount of fatty alcohol, fatty acid (or salt thereof) and fatty alcohol alkoxylate ranges from about 0.1 to about 20% by weight, preferably from about 1 to about 15% by weight, particularly preferably from about 5 to about 15% by weight; in each case based on the weight of the composition.

### Polysaccharides

Optionally, the solid hair conditioning composition disclosed herein comprises at least one polysaccharide d).

Polysaccharides (d) suitable as contemplated herein are composed of more than ten monosaccharide units. Preferred polysaccharides are starches based on α-D-glucose units and starch degradation products such as amylose, amylopectin and dextrins. As contemplated herein, chemically and/or thermally modified starches, e.g. hydroxypropyl starch phosphate, dihydroxypropyl distarch phosphate or the commercial products Dry Flo®, are also particularly advantageous. Dextranes and their derivatives, e.g. dextran sulphate, are also preferred. Also preferred are non-ionic cellulose derivatives, such as methylcellulose, hydroxypropylcellulose, hydroxypropylmethylcellulose or hydroxyethylcellulose, as well as cationic cellulose derivatives, e.g. the commercial products Celquat® and Polymer JR®, and prefers Celquat® H 100, Celquat® L 200 and Polymer JR® 400 (Polyquaternium-10) as well as Polyquaternium-24. Other preferred examples are polysaccharides from fucose units, e.g. the commercial product Fucogel®.

The solid hair conditioning composition disclosed herein suitably comprises:
at least one starch obtained from natural sources (preferably with the starch obtained from natural sources preferably maize, rice, potato or tapioca),
at least one modified starch, and/or
at least one dextrin.

The solid hair conditioning composition disclosed herein preferably comprises:
starch fractions from maize, potatoes, rice, wheat and/or tapioca and/or
modified starches, preferably Hydroxypropyl Starch Phosphate and/or
at least one dextrin, preferably Maltodextrin.

The solid hair conditioning composition disclosed herein preferably comprises starch fractions from maize, potatoes, rice, wheat and/or tapioca and/or at least one dextrin, preferably Maltodextrin. Preferably, the starch fraction is present in excess (by weight) relative to the dextrin component. Preferably, the weight ratio of starch to dextrin ranges from about 10:1 to about 60:1, more preferably from about 20:1 to about 60:1, yet more preferably from about 30:1 to about 50:1.

Preferable starch fractions are those derived from corn, i.e. corn starch. A preferred corn starch is Zea mays (Corn) Starch.

A preferable modified starch is that having the INCI name Hydroxypropyl Starch Phosphate.

A preferable dextrin is that having the INCI name Maltodextrin.

The solid hair conditioning composition disclosed herein preferably comprises a corn starch; maltodextrin; and/or Hydroxypropyl Starch Phosphate, more preferably a corn starch and maltodextrin.

The solid hair conditioning composition disclosed herein preferably comprises a corn starch; maltodextrin; and/or Hydroxypropyl Starch Phosphate, more preferably a corn starch and maltodextrin.

The solid hair conditioning composition disclosed herein suitably comprises from about 10 to about 60%, preferably from about 20 to about 60%, more preferably from about 30 to about 55% and particularly preferably, from about 35 to about 55% by weight of component

d) based on the total weight of the cosmetic composition.

In the context of the present disclosure claimed here, these polysaccharides have proven to be suitable stabilizers. Their use makes it possible to provide ready-made consumer products that maintain their properties and appearance over a long period of time and under various environmental conditions.

### Cationic Polymers

Optionally, the solid hair conditioning composition disclosed herein comprises at least one cationic polymer.

Cationic polymers have conditioning properties, i.e. they give a pleasant feeling to the skin or hair and thus offer added value. They can be used in the context of the present disclosure without significantly impairing the cleaning performance.

Suitable cationic care polymers are, for example:
quaternized cellulose polymers, especially polyquaternium-10, as they are commercially available under the names Celquat® and Polymer JR®,
hydrophobically modified cellulose derivatives, for example the cationic polymers marketed under the trade name SoftCat®,
cationic alkyl polyglycosides,
cationized honey, the commercial product Honeyquat® 50,
cationic guar derivatives, such as the products marketed under the trade names Cosmedia®Guar, N-Hance® and Jaguar® in particular,
polymeric dimethyldiallylammonium salts and their copolymers with esters and amides of acrylic acid and methacrylic acid, in particular polyquaternium-6 and polyquaternium-7. The products commercially available under the names Merquat® 100 (poly(dimethyldiallylammonium chloride)) and Merquat®550 (dimethyldiallylammonium chloride-acrylamide copolymer) are examples of such cationic polymers,
Copolymers of vinylpyrrolidone with quaternized derivatives of dialkylaminoalkyl acrylate and methacrylate, such as vinylpyrrolidone-dimethylaminoethyl methacrylate copolymers quaternized with diethyl sulfate. Such compounds are commercially available under the names Gafquat®734 and Gafquat®755
vinylpyrrolidone-vinylimidazolium methochloride copolymers as offered under the names Luviquat® FC 370, FC 550, FC 905 and HM 552, - quaternated polyvinyl alcohol,
and the polymers known as polyquaternium 2, polyquaternium 17, polyquaternium 18, polyquaternium-24, polyquaternium 27, polyquaternium-32, polyquaternium-37, polyquaternium 74 and polyquaternium 89.

Particularly preferred cationic polymers are quaternized cellulose polymers, hydrophobically modified quaternized cellulose polymers, cationic guar derivatives and/or cationic polymers based on acrylic acid (derivative), which are particularly preferably selected from the guar hydroxypropyltrimonium chloride, polyquaternium-6, polyquaternium-7, polyquaternium-10, polyquaternium-37 and/or polyquaternium-67 known by the INCI designations. Cationic polysaccharide polymers, especially guar hydroxypropyltrimonium chlorides, are particularly preferred in the sense of this present disclosure.

Cationic polymers obtained from natural sources (in addition to guar), such as a cationic inulin polymer, are also particularly preferred. The content of these specific polymer types is not only advantageous for improving hair care properties, but it has also been found that these polymers in combination with other cationic polymers do not cause an over-conditioning effect even after regular use.

Inulin is a polysaccharide that belongs to the group of fructans. In addition to a terminal glucose building block, it comprises up to 60 fructose monomers in the chain, each of which is linked to one another via β-2,1-glycosidic bonds. Inulin may be obtained from the leaves, roots, fruits and/or flowers of composite and/or umbelliferous plants such as Jerusalem artichokes, artichokes and/or parsnips.

Cationic inulin polymers particularly suitable as contemplated herein are cationically modified by reacting hydroxyl groups of the fructose building blocks with reactive quaternary ammonium compounds. Suitable quaternary ammonium compounds are preferably compounds of the following formula

N+(R¹R²R³R⁴)X⁻

in which R¹, R² and R³ are methyl or ethyl groups and R⁴ is an epoxy R⁵ or a halohydrin group Y—CH2-CH(OH)—R⁵—, R⁵ is a C1-C3-alkylene group, Y is a halide and X is an anion such as Cl⁻, Br⁻, I⁻ or HSO₄⁻. Particularly suitable cationic inulin polymers (b) for the purposes of this present disclosure correspond to the formula

R—O—CH₂—CH(OH)—R⁵—N⁺(R¹R²R³)X⁻,

wherein R is inulin and the other radicals have the same meaning as above.

In a particularly preferred form, the compositions as contemplated herein contain cationic inulin polymers which have been cationically modified with cationic hydroxy-C1-C3-alkyl-trialkylammonium groups, in particular with hydroxypropyltrimethylammonium groups. Within this design, cationic inulin polymers known under the INCI designation Hydroxypropyltrimonium Inulin are commercially available (for example, Quatin 1280 TQ-D marketed by Cosun Biobased Products).

The cationic degree of substitution of cationic inulin polymers, in particular cationic inulins known as hydroxypropyltrimonium inulin (INCI), can be varied and adjusted as required. For the use in the invented cosmetic compositions, it has proved to be particularly preferred if such cationically modified inulin polymers exhibit a higher degree of cationic modification (higher cationic degree of substitution), because this allows better coacervate formation and ultimately better care performance to be achieved in the preparations.

In a particularly preferred form, the cationic inulin polymer included in the cosmetic compositions as contemplated herein has a cationic charge density >about 1.5 meq/g, more preferably >about 2.0 meq/g, especially preferred >about 2.5 meq/g, very especially preferred >about 3.0 meq/g and especially >about 3.5 meq/g. Within this design it is particularly preferred if cationic inulin polymers known under the INCI designation hydroxypropyltrimonium inulin have b) a cationic charge density >about 1.5 meq/g, more preferably >about 2.0 meq/g, especially preferred >about 2.5 meq/g, especially preferred >about 3.0 meq/g and especially >about 3.5 meq/g.

In another particularly preferred form, the cationic inulin polymer included in the cosmetic compositions as contemplated herein has b) an average molecular weight of from about 2,000 to about 50,000 g/mol, more preferably from about 2,500 to 40,000 g/mol, more preferably from about 3,000 to about 30,000 g/mol, more preferably from about 3,500 to about 20,000 g/mol and in particular from about 4,000 to about 10,000 g/mol. Within this design it is particularly preferred if cationic inulin polymers known under the INCI designation hydroxypropyltrimonium inulin have b) an average molecular weight of from about 2,000 to about 50,000 g/mol, more preferably from about 2,500 to about 40,000 g/mol, more preferably from about 3,000 to about 30,000 g/mol, more preferably from about 3,500 to about 20,000 g/mol and in particular from about 4,000 to about 10,000 g/mol.

The cationic inulin polymer(s)—preferably known as hydroxypropyltrimonium inulin under the INCI name—are preferably used in the solid compositions disclosed herein in an amount of from about 0.01 to about 5.00% by wt. %, more preferably from about 0.02 to about 4.00 wt. %, particularly preferably from about 0.03 to about 3.00 wt. %, very particularly preferably from about 0.04 to about 2.50 wt. % and in particular from about 0.05 to about 2.00 wt. % (based on the total weight of the composition).

In another preferred form, the cosmetic compositions as contemplated herein contain, instead of the cationic inulin polymer or in addition to the cationic inulin polymer, at least one other cationic polymer selected from cationic polymers of natural origin, preferably cationic polygalactomannan derivatives.

Galactomannans are polysaccharides consisting of combinations of mannose and galactose monomers in different contents. The mannose units are connected to each other via β(1-4)-glycosidic bonds; the galactose units via α(1-6)-bonds. The ratio of mannose to galactose monomers varies according to the type and origin of the plant and the temperature at which it grew. In Greek fenugreek gum, the mannose-galactose ratio is about 1:1 (corresponding to a monomer mannose to a monomer galactose); in guar gum, about 2:1; in tara gum, about 3:1; in locust bean gum, about 4:1; and in cassia gum, about 5:1. All galactomannans from these sources are suitable for cationic modification and use as polymers in the present disclosure cosmetic compositions. Guar gum and/or cassia gum are particularly suitable for use in cosmetic products as contemplated herein.

Like cationic inulin polymers, galactomannans, preferably galactomannans from the above sources, can be cationically modified by reacting the hydroxyl groups of galactomannan polymers with reactive quaternary ammonium compounds. Suitable quaternary ammonium compounds are preferably compounds of the following formula

N+(R¹R²R³R⁴)X⁻

in which R¹, R² and R³ are methyl or ethyl groups and R⁴ is an epoxy R⁵ or a halohydrin group Y—CH²—CH(OH)—R⁵—, R⁵ is a C1-C3-alkylene group, Y is a halide and X is an anion such as Cl⁻, Br⁻, I⁻ or HSO4⁻. Particularly suitable cationic galactomannan polymers within the meaning of this present disclosure correspond to the formula

R—O—CH₂—CH(OH)—R⁵—N⁺(R¹R²R³)X⁻,

wherein R represents the respective galactomannan and the other radicals have the same meaning as defined above.

In a particularly preferred form, the compositions as contemplated herein therefore contain cationic galactomannan polymers which have been cationically modified with cationic hydroxy-C1-C3-alkyl-trialkylammonium groups, in particular with hydroxypropyltrimethylammonium groups. Galactomannan polymers which have been cationically modified with cationic hydroxy-C1-C3-alkyl-trialkylammonium groups, in particular with hydroxypropyltrimethylammonium groups, and which originate from guar gum and/or cassia gum are particularly preferred within this design.

In a particularly preferable form, the cosmetic compositions which have been invented contain at least one of the compounds known as Guar Hydroxypropyltrimonium Chloride, Hydroxypropyl Guar Hydroxypropyltrimonium Chloride and/or Cassia Hydroxypropyltrimonium Chloride, as a polygalactomannan derivative, as the INCI designations.

Guar and cassia polymers known under these INCI designations are available from various suppliers, for example under the designations Jaguar®, N-Hance®, Polycare®, Clearhance®, Activsoft®, Guarquat®, Vida-Care®. Jaguar® C-162, Jaguar® C500, Jaguar® Styl 100, N-Hance® 3196, N-Hance® HPCG 1000, Activsoft® C17, Guarquat® C130 KC, Guarquat® CP500 KC, Vida-Care® GHTC and/or Polycare® Split Therapy are specific examples of cationic polymers of natural origin that are particularly suitable as contemplated herein.

Cationic polymers are used in the solid compositions disclosed herein (based on their total weight) preferably in amounts of from about 0.01 to about 2.00 wt. %, more preferably from about 0.02 to about 0.90 wt. %, more preferably from about 0.03 to about 0.80 wt. %, more particularly preferably from about 0.04 to about 0.70 wt. % and in particular from about 0.05 to about 0.60 wt. %.

### Further Optional Components

In addition to the ingredients described above, the cosmetic compositions as contemplated herein may contain at least one active ingredient advantageously selected from the group comprising plant extracts, liquid oils, bitter substances, humectants, protein hydrolysates, perfumes, UV filters, structuring agents such as maleic acid, dyes for staining the composition, Active ingredients such as bisabolol and/or allantoin, antioxidants, preservatives such as sodium benzoate or salicylic acid, additional viscosity regulators such as salts (NaCl) or polymers, and pH regulators such as α and β hydroxycarboxylic acids such as citric acid, lactic acid, malic acid, glycolic acid, and/or bases such as alkanolamines and/or sodium hydroxide).

Suitable plant extracts are extracts that can be produced from all parts of a plant. Usually, these extracts are produced by extracting the entire plant. However, in some cases it may also be preferable to produce extracts exclusively from the flowers and/or leaves of the plant. The most suitable extracts are Paeonia lactiflora, Rosa damascena Flower, Malus domestica Fruit, Argania spinosa Shell Powder, Laminaria saccharina, Cannabis sativa, Green Tea, Oak Bark, Nettle, Hamamelis, Hops, Camomile, Burdock Root, Horsetail, Hawthorn, Linden Flower, Litchi, Almond, Aloe Vera, Spruce Needle, Horse Chestnut, Sandalwood, juniper, coconut, mango, apricot, lime, wheat, kiwi, melon, orange, grapefruit, sage, rosemary, birch, mallow, cuckoo flower, quendel, yarrow, Thyme, lemon balm, hemlock, coltsfoot, marshmallow, ginseng, ginger root, Echinacea purpurea, Olea europea, Boerhavia diffusa roots, Foeniculum vulgaris and Apim graveolens. The extracts from Paeonia lactiflora, Rosa damascena Flower, Malus domestica Fruit, Argania spinosa Shell Powder, Laminaria saccharina, Cannabis sativa, Green Tea, Nettle, Hamamelis, Camomile, Aloe Vera, Ginseng, Echinacea purpurea, Olea europea and/or Boerhavia diffusa roots are particularly preferred for use in the compositions as contemplated herein. Water, alcohols and their mixtures can be used as extraction agents for the production of the plant extracts mentioned. Among the alcohols, lower alcohols such as ethanol and isopropanol are preferred, but in particular polyhydric alcohols such as ethylene glycol and propylene glycol, both as the sole extraction agent and mixed with water. Plant extracts based on water/propylene glycol in a ratio of from about 1:10 to about 10:1 have proven to be particularly suitable. The plant extracts can be used in both pure and diluted form. If they are used in diluted form, they usually contain approx. 2-80 wt. % active substance and as solvent the extraction agent or mixture of extraction agents used in their extraction. The plant extracts can be used in the hair treatment products as contemplated herein (based on the total weight of the products) preferably in an amount of from about 0.01 to about 10% by weight, more preferably from about 0.05 to about 7.5% by weight and in particular from about 0.1 to about 5% by weight.

Under suitable pH control agents, particularly preferred for use in the compositions as contemplated herein are citric acid, lactic acid, malic acid, glycolic acid, in particular citric acid and/or lactic acid.

Examples of oils (other than the solid oil component (a) in the composition of the present disclosure) include liquid oils, hydrocarbon oils, and silicone oils. It is preferred that the compositions disclosed herein contain no silicone oils, and no silicones in any form (i.e. that they are silicone-free). It is particularly preferred that the compositions disclosed herein are silicone-free and contain no hydrocarbon oils. Thus, the preferred liquid oils and particularly those of natural origin.

Suitable oils are perfume oils and/or vegetable triglyceride oils, such as coconut oil, (sweet) almond oil, walnut oil, peach kernel oil, apricot kernel oil, avocado oil, tea tree oil, soya oil, cotton seed oil, sesame oil, sunflower oil, tsubaki oil, evening primrose oil, rice bran oil, palm oil, Palm seed oil, mango seed oil, cranberry oil, sea buckthorn oil, lady's smock oil, safflower oil, macadamia nut oil, grape seed oil, amaranth seed oil, argan oil, bamboo oil, olive oil, Wheat germ oil, pumpkin seed oil, mallow oil, hazelnut oil, safflower oil, maize oil, olive oil, rapeseed oil, canola oil, sasanqua oil, jojoba oil, rambutan oil, marula oil and/or quinoa oil.

Especially preferred are apricot kernel oil, argan oil, jojoba oil, marula oil, macadamia nut oil, pumpkin seed oil, amaranth seed oil, quinoa oil, soya oil, cottonseed oil, sunflower oil, palm oil, palm kernel oil, linseed oil, almond oil, corn oil, olive oil, rapeseed oil, sesame oil, soya oil, safflower oil, wheat germ oil, peach kernel oil, cranberry oil, sea buckthorn oil and/or coconut oil.

Especially preferred are apricot kernel oil, argan oil, jojoba oil, marula oil, almond oil, olive oil, coconut oil and/or sunflower oil.

The oil(s) may be used in the compositions disclosed herein in a proportion by weight of from about 0.01 to about 10% by weight, more preferably from about 0.05 to about 7% by weight, more preferably from about 0.10 to about 5% by weight of the total weight of the compositions.

The solid compositions disclosed herein may comprise from about 0.001 to about 0.01% by weight of at least one bitter substance, preferably a compound known as Denatonium Benzoate INCI.

A bitter substance is particularly important in cosmetics, household products etc., which are made up in such a way that their shape, colour, haptics etc. appeal to small children or babies and encourage them to play, which could also lead to swallowing. A bitter substance prevents this. Denatonium Benzoate is an extremely strong bitter substance and is therefore particularly effective even at very low concentrations. Furthermore, it is not associated with any known adverse effects.

The present disclosure also concerns a solid hair conditioning composition as disclosed herein with a Shore A hardness value of from about 10 to about 30 and/or a Shore AO hardness value of from about 5 to about 60.

Such a hardness range is perceived as pleasant by users. In this area it is possible to extract a small amount of solid hair conditioning composition for one application from a crucible intended for several applications with the hands and without any further aids.

If a Shore A hardness is indicated in the context of this notification, this means that the hardness was determined by measurement using a truncated cone tip needle with a diameter of 1.3 millimetres at an opening angle of 35° on the face of the truncated cone. If a Shore AO hardness is given, it was measured by a needle with a spherically rounded tip with a radius of 2.5 mm of the ball segment. A durometer was “placed” on the product without additional pressure and the maximum value was read. A multiple measurement (at least 3 measurements) was performed and the mean value was given. A manual analog durometer from Sauter GmbH was used. Unless expressly stated otherwise, the equipment has been operated in accordance with the instructions Version 1.2, as of August 2014, which accompanies this equipment and states that it complies with DIN 53505, ASTM D2240 and ISO 868.

### METHODS AND USES

The present disclosure also concerns a solid hair conditioning composition as previously described for the care of human hair, in particular for use after hair cleansing as a leave-on or rinse-off composition. The compositions may also be useful in hair treatments, and hair packs.

The terms ‘leave-on’ and ‘rinse-off’ mean that the composition is either left in the hair for a relatively short time, for example for less than one minute if necessary, or for a few minutes or one hour, until it is rinsed out again, or that the composition remains in the hair until the next hair wash, which may be a few days. Both have certain advantages. If the composition remains on the hair for a long time, the full care potential of all ingredients can be exploited, whereas a composition to be rinsed out in a short time can also contain ingredients that have a good care effect but whose prolonged retention in the hair would be unpleasant.

Preferred in the sense of the present disclosure are rinse-off compositions.

The present disclosure also concerns a solid hair conditioning composition as described above in the form of a bar or a stick, or as a “portion”.

As can be seen from the production processes and applications revealed herein, a bar or stick is well suited for multiple use by the user. The present disclosure therefore represents the solid hair conditioning composition as described above in the form of a multiple-use bar, or as a multi-use stick. A bar may be generally described as a product that is provided as is. A stick (also known as a “pen”) can be manufactured such that one end of the stick may remain in a wrapper or package during use, so that a user is not faced with the problem of having to hold a slippery piece of solid cosmetic preparation. The design can, for example, be chosen as in the case of a shaving soap, in which there is usually a fixed base on one side of the stick and the stick itself is surrounded by a gradually removable, easily manually tearable cover, or it can resemble more a deodorant stick, i.e. it can include a fixed cover also on the outer circumferential surface of the stick and a mechanism to gradually push the pen forward in the cover, so that it protrudes a little beyond the opening again and again until it is completely worn away through repeated use. Also the packaging for a transport, for example during a journey, is quite simple with a stick, since a cap surrounding or covering the stick can easily be attached. Pencils would be problematic with a conventional conditioner formulation because the material removal at the relatively small surface would be too slow. With the inventive compositions, they can be easily realized and the speed of material removal in the application meets the expectations of the users without, on the other hand, leading to a wasteful use through excessive removal.

The “portion” can be dimensioned so that it can be used either singly or several times. Particularly portions are dimensioned in such a way that they are only intended to be used once. We refer herein to such a product form as a “monoportion”.

These packaging forms each have certain advantages. A monoportion still resembles in a way traditionally used cosmetics and is often preferred by less experimental users. A bar/stick is easier to transport, as it is usually provided with a cap or cover and is easier to hold, as it can be gripped by its base, cover or outer packaging.

The present disclosure further concerns a process for preparing the solid hair conditioning composition as described above, comprising the process:

a) Mixing of all ingredients with sufficient heating and agitation to ensure that all ingredients are melted and/or evenly distributed,
b) Allow the resulting mixture to cool and solidify.

Such a process makes a uniformly mixed solid hair conditioning composition as described above actually available and its advantages tangible.

The present disclosure further concerns a process for preparing the solid hair conditioning composition as described above, comprising the process:

a) Mixing of all ingredients with sufficient heating and agitation to ensure that all ingredients are melted and/or evenly distributed,
b) Pour the resulting mixture into a bar/stick mould,
c) Allow the mixture to cool and solidify.

Such a process makes a uniformly mixed solid hair conditioning composition in stick or piece form as described above actually available and its related advantages as described above can be experienced.

The present disclosure further concerns a process for preparing the solid hair conditioning composition as described above, comprising the process:

a) Mixing of all ingredients with sufficient heating and agitation to ensure that all ingredients are melted and/or evenly distributed,
b) Pour the resulting mixture into a mould,
c) Allow the mixture to cool and solidify.

Such a process makes a uniformly blended solid hair conditioning composition actually available in a particular manner as described above and allows its related benefits as described above to be experienced.

The present disclosure also concerns a hair treatment process, preferably hair conditioning, in which a solid hair conditioning composition is mixed with water and applied to the hair as previously described or in which the solid hair conditioning composition is applied directly to wet hair and worked in.

This procedure allows the user to experience the advantages described above and thus represents an enrichment not only for personal hygiene and care but also for sensory enrichment.

The present disclosure also concerns the use of a solid hair conditioning composition as described above to treat, preferably condition and/or care for the hair.

This use makes the advantages described above tangible for the user and thus represents an enrichment not only for personal hygiene and care but also for sensory enrichment.

As has become apparent from the previously described design forms and their advantages, the process and packaging aspects are important for this present disclosure. They are discussed in more detail below.

In a manufacturing process as contemplated herein, for example, all ingredients are put into a heatable container, such as, on a laboratory scale, into a suitable vessel in a water bath or on a heating plate, on a production scale rather into a closed and pressurizable vessel, and mixed and heated, for example at 75° C., until all ingredients are sufficiently mixed. In such a process, different temperature steps can also be carried out. For example, components that can be mixed homogeneously even at a relatively low temperature can be mixed first. This can be done at from about 40° C. to about 50° C. It can also be advantageous to mix in certain ingredients at higher temperatures, for example at from about 85° C. to about 90° C. For this purpose, a procedure as contemplated herein may include one or more steps in this temperature range. One or more steps can then be carried out at a lower temperature, during which further components are mixed in. Typically, the compositions as contemplated herein solidify at about 65° C., so that certain process steps, such as mixing and extruding the finished mixtures below such a temperature level, are not reasonably possible.

When an inventive composition is extruded, the available shape can be determined by a shape of the die closing the extruder. The solidifying mixture can be filled into moulds, portioned onto a base or produced endlessly and cut and portioned at the nozzle or subsequently.

The extrusion processes described above can also be used to produce interesting shapes reminiscent of biscuits. Specially shaped dies on the extrusion die make it possible to create a wide variety of shapes, such as a heart or clover leaf. An extruded strand thus obtainable can then be cut into pieces or slices, providing emotionally appealing small portions of the present disclosure's solid cosmetic compositions. It is also possible to roll out an extruded strand or other form of a composition as contemplated herein and then, by punching or cutting or cutting, produce pieces which are similar to cookie cutters in terms of forming characteristics.

It is also possible to fill the present disclosure's solid cosmetic compositions into a crucible, for example made of glass. Since the strength of these compositions is in a range that allows manual removal of a portion from a crucible intended for several applications, without the use of a tool. If the composition was foamed in a crucible during its production, this results in a particularly interesting feeling.

It is also possible to make up a solid cosmetic composition reminiscent of a piece of paper, a foil or a wafer, as contemplated herein, and this brings with it a new and pleasant feeling during application. Since the thickness of the layer is small with this type of packaging, short dissolving times can be achieved, which are beneficial for impatient users and do not favour wasteful use of water. A product made up in this way can be placed on the market in a packaging unit in which a large number of sheets or platelets are placed in a small, possibly subdivided carton so that a single removal is possible.

The following example is intended to assist one skilled in the art to better understand and practice the present disclosure. The scope of the present disclosure is not limited by the example but is defined in the appended claims. All parts and percentages are based on weight unless otherwise stated.

### Example

| INCI Ingredient | Weight % |
|---|---|
| Zea Mays (Corn) Starch¹ | 47.5 |
| Butyrospermum Parkii (Shea) Butter² | 20.2 |
| Sodium bicarbonate | 10.1 |
| Glycerin | 10.0 |
| Aqua | 6.1 |
| Quaternium-87³ | 2.3 |
| Cetrimonium Chloride⁴ | 2.0 |
| Maltodextrin⁵ | 1.0 |
| Propylene Glycol | 0.76 |
| Citric Acid | 0.04 |

¹Maisita 9040
²Cetiol SB 45
³Varisoft W 575 PG
⁴Dehyquart A CA
⁵Agenamalt 20.225 Maltodextrin DE15

Dehyquart A CA was heated in a drum to 40° C. to 50° C. and mixed in case of uneven distribution of its ingredients. After mixing to homogeneity, the other ingredients were added, ensuring with each addition that the liquid was mixed again to homogeneity and at a suitable temperature to maintain the liquid mixture (around 80° C. to 90° C.). After that, the temperature was no longer kept active at 85° C. to 90° C. but it was only ensured that it did not fall to 70° C. or less. This was followed by pouring the resulting mixture into a bar mould. The mixture was then allowed to cool and solidify.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A solid hair conditioning composition, comprising
(a) from about 10 wt % to about 70 wt % of at least one fat or oil having a melting point above about 20° C.,
(b) from about 3% to about 50 wt % of at least one cationic surfactant, and
(c) from about 0.5 wt % to about 20 wt % of at least one polyhydric alcohol and/or polyethylene glycol,
wherein the water content is less than about 20 wt %, and wherein all amounts are based on the total weight of the solid hair conditioning composition.

2. The solid hair conditioning composition according to claim 1, wherein component (a) is a solid vegetable butter.

3. The solid hair conditioning composition according to claim 1, wherein component (a) is selected from Butyrospermum parkii (Shea) Butter, Theobroma cacao (Cocoa) Seed Butter, Cocos nucifera (Coconut) Oil, Elaeis guineensis (Palm) kernel oil, Elaeis guineensis (Palm) Kernel Oil, Japan Wax, Synthetic Japan Wax, Ricinus communis (Castor) Seed Oil, Simmondsia chinensis (Jojoba) Butter and mixtures thereof.

4. The solid hair conditioning composition according to claim 1, wherein the component (c) is selected from butylene glycol, propylene glycol, erythritol, maltitol, glycerin, polyethylene glycol with a molecular weight of from about 3,000 to about 300,000, and mixtures thereof.

5. The solid hair conditioning composition according to claim 1, having a water content less than or equal to about 10 wt %, based on the total weight of the solid hair conditioning composition.

6. The solid hair conditioning composition according to claim 1, further comprising from about 10 to about 60% by weight of at least one polysaccharide, based on the total weight of the solid hair conditioning composition.

7. A solid hair conditioning composition according to claim 1, in the form of a bar.

8. The solid hair conditioning composition according to claim 7, having a Shore A hardness value of from about 10 to about 30.

9. A method for preparing the solid hair conditioning composition according to claim 1, the method comprising
(i) mixing all the components with sufficient heating and agitation to ensure all the components are melted and/or uniformly dispersed,
(ii) pouring the resultant liquid into a mold or directly into packaging, and
(iii) allowing the liquid to cool into a solid.

10. A method for treating hair with the solid hair conditioning composition according to claim 1 comprising:
mixing the solid hair conditioning composition with water and applying to the hair, or applying the solid hair conditioning composition directly to wet hair.

11. A solid hair conditioning composition according to claim 1, wherein component (a) comprises Butyrospermum parkii (Shea) Butter.

12. The solid hair conditioning composition according to claim 6, comprising from about 20 to about 60% by weight of the at least one polysaccharide, based on the total weight of the solid hair conditioning composition.

13. A solid hair conditioning composition according to claim 1, wherein the content of component (a) is from about 10 wt % to about 30 wt %, based on the total weight of the solid hair conditioning composition.

14. The solid hair conditioning composition according to claim 1, comprising as cationic surfactant (b) at least one compound selected from the group of:
i. alkylquats,
ii. esterquats,
iii. quaternary imidazolines,
iv. amidoamines and/or cationized amidoamines and
v. mixtures thereof.

15. A solid hair conditioning composition according to claim 14, comprising at least one cationic surfactant b) from group i and at least one cationic surfactant b) from group iii.

16. A solid hair conditioning composition according to claim 14, comprising as cationic surfactant (b): i) cetrimonium chloride and/or behentrimonium chloride; and iii) Quaternium-87.

17. The solid hair conditioning composition according to claim 1, wherein the content of the component (b) is from about 3 wt % to about 10 wt %, based on the total weight of the solid hair conditioning composition.

18. The solid hair conditioning composition according to claim 1, wherein the at least one polyhydric alcohol and/or polyethylene glycol comprises a mixture of glycerin and a glycol.

19. The solid hair conditioning composition according to claim 1, wherein the content of component (c) is from about 5 wt % to about 15 wt %, based on the total weight of the solid hair conditioning composition.

20. The solid hair conditioning composition according to claim 1, wherein:
component (a) comprises Butyrospermum parkii (Shea) Butter;
the content of component (a) is from about 10 wt % to about 30 wt %;
cationic surfactant (b) comprises i) cetrimonium chloride and/or behentrimonium chloride and iii) Quaternium-87;
the content of the component (b) is from about 3 wt % to about 10 wt %;
the at least one polyhydric alcohol and/or polyethylene glycol comprises a mixture of glycerin and a glycol;
the content of component (c) is from about 5 wt % to about 15 wt %; and
the composition further comprises from about 20 to about 60% by weight of at least one polysaccharide;
wherein all amounts are based on the total weight of the solid hair conditioning composition.
